(19) United States
(12) Patent Application Publication
(10) Pub. No.: US 2005/0004217 A1
(43) Pub. Date: Jan. 6, 2005

(54) Accelerator of collagen production

(76) Inventors: Tomoko Yamamoto, Hiratsuka-shi (JP); Ken-ichi Yamamoto, Hiratsuka-shi (JP)

Correspondence Address: KUBOVCIK & KUBOVCIK, SUITE 710, 900 17TH STREET NW, WASHINGTON, DC, 20006, US

(21) Appl. No.: 10/870,495

(22) Filed: Jun. 18, 2004

(30) Foreign Application Priority Data
Jun. 19, 2003 (JP) 2003-175372

(51) Int. Cl. A61K 31/20; A61K 35/78

(52) U.S. Cl. 514/559; 424/725

(57) ABSTRACT

An accelerator of collagen production containing a compound represented by formula (I) or a salt thereof 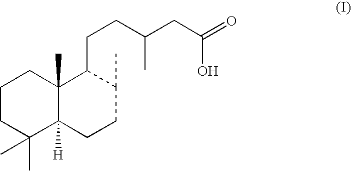 wherein the three dotted lines represent two single bonds and one double bond.

### BACKGROUND OF THE INVENTION

1. Field of the Invention

The present invention relates to a collagen production accelerator comprising labdenoic acid compounds. Further, the present invention relates to an agent for external application onto the skin and oral cavity compositions containing the above-mentioned compounds.

2. Prior Art

The living body is constituted by cells and a matrix outside the cells which fills the spaces between cells. Among the elements constituting the extracellular matrix, collagen is the main constituent of fiber and makes up about one-third of the total mass of body protein. Collagen exists in all the internal organs of the body, such as the heart, liver, and muscles, and the skin, bone, cartilage, tendons, blood vessels, etc. have especially high collagen contents.

Collagen not only supports the structure of tissues but affects the functions of the living body by having influences on shape, metabolism, adhesion, etc. of various cells. Thus, decreasing of collagen in connection with age has also been reported and since collagen has an important role in a living body, it is also considered that this is the main cause of wrinkles and flabby skin. On the other hand, ascorbic acid and its derivatives, retinoic acid, insulin, growth hormone, TGF-β, estrogen, etc. are known as substances that promote biosynthesis of collagen. (For example, see Japanese Patent Application JP06-157232-A or JP09-241125-A). However, the collagen production accelerators have many deficiencies, such as stability, side effects, etc., and a new collagen production accelerator has been desired.

### SUMMARY OF THE INVENTION

An object of the present invention is to provide compounds which facilitate collagen production, are safe, and are free of side effects.

As a result of their eager study to solve these problems, the present inventors found that extracts with hot water, or extracts with ethanol, hexane, etc. of stems, branches, leaves, etc. of Cistus ladaniferus L., Cistus creticus L., Cistus monoperiensis L., Cistus salvifolius, etc., have a strong acceleratory activity on the production of collagen, and that this action is based on labdenoic acid. The inventors found further that labd-7-en-15-oic acid, labd-8(17)-en-15-oic acid, and labd-8-en-15-oic acid are contained as the main components of the above extracts, and that their salts have a beneficial effect of accelerating production of collagen, and as a result of additional examination, the present invention was completed at last.

The present invention includes the following.
1. An accelerator of collagen production containing a compound represented by formula (I) or a salt thereof 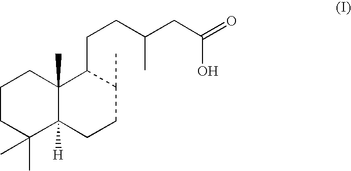
   wherein the three dotted lines represent two single bonds and one double bond.
2. The accelerator of 1, wherein the compound represented by formula (I) is obtainable from an extract of a cistaceous plant.
3. The accelerator of 2, wherein the cistaceous plant is selected from the group consisting of Cistus ladaniferus L., Cistus creticus L., Cistus monoperiensis L. and Cistus salvifoliud.
4. The accelerator of 1, wherein the compound represented by formula (I) is synthesized by a chemical process.
5. The accelerator of 4, wherein the compound represented by formula (I) is synthesized from sclareol or manool.
6. An agent for external application onto the skin containing a compound represented by formula (I) or a salt thereof.
7. An oral cavity composition containing a compound represented by formula (I) or a salt thereof.

### DETAILED DESCRIPTION OF THE INVENTION

The above compounds are those known in the art and processes for their production are also known. For example, labdenoic acid is a component in labdanum gum extracted from Cistus ladaniferus (J. Chem. Soc., 1956, 4259-4262), and labd-8(17)-en-15-oic acid (eperuic acid) and labd-8-en-15-oic acid are obtained by chemically treating labdenoic acid (J. Chem. Soc., 1956, 4262-4271). Further, it is reported that eperuic acid is a component in a resin derived from an Eperua falcata tree of the Leguminosae (J. Chem. Soc., 1955, 658-662), and labd-7-en-15-oic acid (cativic acid) is a component in a resin from Prioria copaifera G. tree of the Leguminosae (J. Am. Chem. Soc., Vol. 79, 1201-1205, 1957). Hereinafter, these compounds and the salts of labd-8(17)-en-15-oic acid, labd-8-en-15-oic acid, and labd-7-en-15-oic acid may be referred to collectively as labdenoic derivatives.

Although the plants used for preparing the compounds defined in the present invention are not particularly limited insofar as they are plants containing said compounds, it is particularly advantageous to employ Cistus ladaniferus L., Cistus creticus L., Cistus monoperiensis L., and Cistus salvifolius plants (Cistaceae family). These are used alone or in combinations thereof. The part of the plant used is not particularly limited, and use is made of leaves, branches, stems, barks, etc. These may be used just after being harvested or after being dried.

Preferably, the method of extracting the desired compounds from said plants makes use of one or more solvents selected from the group consisting of water, lower alcohols, petroleum ethers and hydrocarbons. The lower alcohols are those containing 1 to 4 carbon atoms, preferably methanol, ethanol, etc.

The petroleum ether used may be a commercial product having 30˜70° C./1 atm as a boiling point.

The hydrocarbon solvents are aliphatic hydrocarbons, alicyclic hydrocarbons and aromatic hydrocarbons which are liquid at ordinary temperatures and, preferably, are aliphatic hydrocarbons and aromatic hydrocarbons which are liquid at ordinary temperatures, among which hexane and toluene are particularly preferable.

Although the operation of extraction differs depending on the plant and solvent used, usually, divided pieces of the plant are immersed in the solvent, optionally under gentle stirring, at room temperature to a temperature of 50° C.

Further, a soxhlet extractor known in the art may also be used.

The time required for extraction is usually 3 to 48 hours.

Alternatively, a method of steam distillation or boiling in hot water after leaves, branches or stems of the plant are disrupted or broken may also be adopted in the present invention. In this case, gum which floats on the water upon steam distillation or hot-water extraction is removed and then separated from insolubles by means of solvent extraction.

Further, commercially available products obtained from the above plants by any of the methods described above may be used.

The crude extract thus obtained contains 25 to 35% labdenoic acid. This crude extract itself may be used as an accelerator of collagen production.

Further, the above crude extract or a commercially available extract may be subjected to molecular distillation under reduced pressure at 13.3 to 66.7 Pa whereby a fraction at 160 to 230° C. and, preferably, 180 to 220° C. is collected. This fraction, which contains a mixture of labd-7-en-15-oic acid, labd-8(17)-en-15-oic acid and labd-8-en-15-oic acid, may be used as an accelerator of collagen production.

Next, a method of obtaining labdenoic derivatives useful as an accelerator of collagen production of present invention is explained by chemical synthesis. As a synthetic method, although the method of following scheme 1 or scheme 2 is mentioned, for example, it is not limited to these. 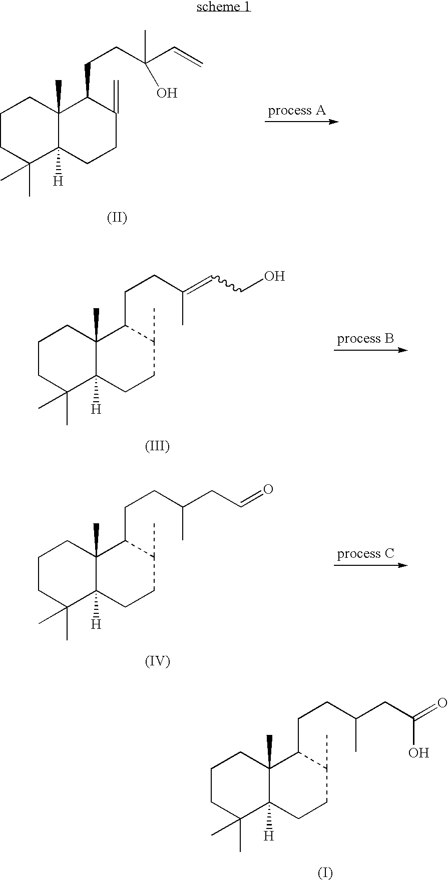
wherein, as for a wavy line, a double bond shows a mixture of E and Z, or E or Z. Three dotted lines represent two single bonds and one double bond. 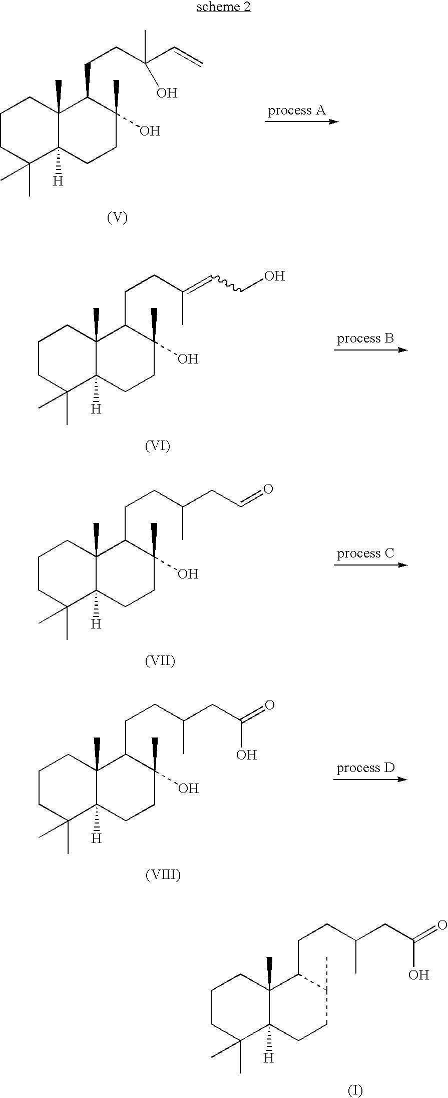
wherein, as for a wavy line, a double bond shows a mixture of E and Z, or E or Z. Three dotted lines represent two single bonds and one double bond.

In scheme 1 and scheme 2, a process A is a production of allyl alcohol compounds (II) and (V) by an allyl rearrangement reaction of manool and sclareol, in an alcohol, in the presence of boric acid, by using a vanadate or molybdate as a catalyst. Thereafter, the said allyl alcohol is converted to an aldehyde compound (III) or (VI) by using a ruthenium-phosphine complex as a catalyst in process B and in process C, and the thus obtained aldehyde is oxidized by an oxidant such as sodium chlorite with an amidesulfuric acid in order to obtain carboxylic acid (I) and (VIII). Further, labdenoic derivatives as an active ingredient of the present invention are obtained by dehydration of the carboxylic acid compound (VIII) by using an acid catalyst in process D.

Since the labdenoic derivatives used by the present invention have a carboxyl group in the molecule, said carboxyl group may be free or a salt. The salt includes, for example, an alkali metal salt such as sodium and potassium, an alkaline earth metal salt such as calcium and magnesium, and an ammonium salt such as ammonium, monomethyl ammonium, dimethyl ammonium, trimethylammonium and dicyclohexyl ammonium.

It is preferable to increase the water solubility in some types of products and, on the contrary, the oil solubility due to free carboxylic acid is advantageous in other types of products, and thus the labdenoic acids used in the present invention may be water-soluble or oil-soluble depending on the needs. A well-known means can easily perform conversion to the salt from the free carboxylic acid by reacting, for example, the above-mentioned alkaline metal hydroxide, alkaline-earth-metals hydroxide, and amine with the carboxylic acid. Conversely, conversion to the free carboxylic acid from the salt can be easily performed by reacting an acid, such as hydrogen chloride and sulfuric acid, with the salt.

Thus obtained labdenoic derivatives are useful for accelerating collagen production.

Further, these derivatives can be incorporated into an agent for external application onto the skin such as a conditioner, a skin cream, an emulsion, a face pack and an ointment, an oral cavity composition such as tooth paste, mouth wash, etc. to give a corresponding agent having a facilitatory effect on collagen production. Further, the compound (I) and its salt of the present invention can be added to other components to prepare an anti-aging agent and anti-wrinkle agent, etc.

The amount of the compound (I) or a salt thereof the present invention incorporated, solely or as a mixture of two or more kinds of them, in various external preparations is usually about 0.001 to 10% by weight, preferably about 0.01 to 5% depending on the types of products and the frequency of use.

Further, the accelerator of collagen production of the present invention can contain not only the labdenoic derivatives as active ingredient(s) but also other ingredients used in agents for usual cosmetics, quasi drug preparations, pharmaceutical preparations, etc. within an effective range of the present invention. For example, it is possible to incorporate surface active agents, oil components, alcohols, moisturizers, thickeners, preservatives, antioxidants, chelating agents, pH adjusters, perfumes, coloring agents, UV absorbers and scatterers, vitamins, amino acids and water.

Hereinafter, some of these ingredients are exemplified.

The surface active agents can be exemplified by nonionic surface active agents such as a lipophilic glyceryl monostearate, a type of self emulsified glyceryl monostearate, polyglyceryl monostearate, sorbitan monooleate, polyethyleneglycol monostearate, polyoxysorbitan monooleate, polyoxyethylenecetylether, polyoxyethylene sterol, polyoxyethylene lanoline, polyoxyethylene yellow beeswax and polyoxyethylene hydrogenated castor oil; anionic surface active agents such as sodium stearate, potassium palmitate, sodium cetyl sulfate, sodium lauryl phosphate, sodium lauryl sulfate, triethanolamine palmitate, sodium polyoxyethylene lauryl phosphate and sodium N-acylglutamate; and cationic surface active agents such as stearyl dimethylbenzyl ammonium chloride and stearyl trimethyl ammonium chloride.

The oil components can be exemplified by a plant-derived oil such as castor oil, olive oil, cacao fat, Japan wax, jojoba oil, grape seed oil and avocado oil; an animal fat and oil such as mink oil and egg yolk oil; a wax such as yellow beeswax, spermaceti, lanoline, carnauba wax and candelilla wax; a hydrocarbon such as liquid paraffin, squalane, microcrystalline wax, ceresin wax and Vaseline; natural or synthetic fatty acids such as lauric acid, myristic acid, stearic acid, oleic acid, isostearic acid and behenic acid, natural or synthetic higher alcohols such as cetyl alcohol, stearyl alcohol, 2-hexyl-1-decanol, 2-octyl-1-dodecanol and lauryl alcohol; and esters such as isopropyl myristate, isopropyl palmitate, 2-octyl-1-dodecyl myristate, 2-octyl-1-dodecyl oleate and chorestelyl oleate.

The alcohol compounds can be exemplified by methanol, ethanol, isopropanol, menthol and isopulegol.

The moisturizers can be exemplified by polyols such as glycerine, propylene glycol, 1,2-butandiol, sorbitol, polyglycerine, polyethylene glycol and dipropylene glycol, an NMF (natural moisture factor) compound such as an amino acid, sodium lactate and sodium pyrolidone carboxylate, a water soluble polymer such as hyaluronic acid, mucopolysaccharide and chondroitin sulfate.

The thickeners can be exemplified by a natural polymer such as sodium argininate, a xanthan gum, an aluminum silicate, an extract of equince seed, a tragacanth gum and starch; a semisynthetic polymer such as methyl cellulose, hydroxyethyl cellulose, carboxy-methyl cellulose, fusibility starch and cationic cellulose; and a synthetic polymer such as carboxy-vinyl polymer and polyvinyl alcohol.

The preservatives can be exemplified by benzoate salt, salicylate salt, sorbate salt, dehydroacetate salt, paraoxybenzoate, 2,4,4′-trichloro-2′-hydroxydiphenylether, 3,4,4′-trichlorocarbanilide, benzalkonium chloride, hinokitiol, resorcinol and ethanol.

The anti-oxidants can be exemplified by 2,6-di-tert-butyl-p-cresol, 2,6-di-tert-butyl anisol, nordihydroguaiaretic acid, propyl gallate, ascorbic acid and tocopherol.

The chelating agents can be exemplified by disodium edetate, eyhylenediaminetetraacetate salt, pyrophosphate, hexametaphosphate, citric acid, tartaric acid and gluconic acid.

The pH adjusters can be exemplified by sodium hydroxide, triethanolamine, citric acid, sodium citrate, boric acid, pyroborate and potassium dihydrogenphosphate.

The UV absorbers and scatterers can be exemplified by 2-hydroxy-4-methoxybenzophenone, 2-ethylhexyl 4-dimethylaminobenzoate, 2-ethylhexyl 4-methoxycinnamate, titanium oxide, kaolin and talc.

The vitamins can be exemplified by vitamin A, vitamin B, vitamin C, vitamin D, vitamin E, vitamin F, vitamin K, vitamin P, vitamin U, carnitine, ferulic acid, γ-oryzanol, α-lipoic acid and orotic acid.

The amino acids can be exemplified by glycine, alanine, valine, leucine, isoleucine, serine, threonine, phenylalanine, tyrosine, tryptophan, cystine, cysteine, methionine, proline, hydroxyproline, aspartic acid, glutamic acid, arginine, histidine and lysine.

Some of the above components further improve the effectiveness of external preparations for skin or oral compositions of the present invention by enhancing stability or transdermal absorption of labdenoic acids which are the active ingredient according to the present invention.

In addition, such auxiliary ingredients are not limited to the above compounds. By suitably blending labdenoic derivatives, which are an active compound, and auxiliary ingredients for an agent for external application onto the skin or an oral cavity composition of the present invention, various product forms, such as a conditioner, a cream, a lotion, an emulsion, a face pack, an ointment, a tooth paste and a mouthwash are possible.

Moreover, labdenoic derivatives used in the present invention are useful also as an accelerator of collagen production in oral medicines (e.g., tablet, powder, granule) or parenteral medicines (e.g., parenteral injection). Such medical supplies can be easily manufactured by mixing the derivatives with well-known diluents, extenders, etc. Furthermore, the labdenoic derivatives of the present invention are useful as food additives for health food manufacture, and can be added to various foods. Therefore, medicines and foods containing the above-mentioned labdenoic derivatives should be considered as being within the scope of the present invention.

Excipients to be combined with the labdenoic acids are selected from those known in the art and examples thereof include, sugar alcohols such as D-sorbitol, D-mannitol and xylitol; solid diluents such as crystalline cellulose, carmellose sodium, calcium hydrogen phosphate, wheat starch, rice starch, corn starch, potato starch, dextrin, β-cyclodextrin, light anhydrous silicic acid, titanium oxide and magnesium aluminometasilicate; and liquid diluents such as injectable solutions, distilled water, etc. and the like.

Although the amount of labdenoic derivatives used in medicines, health foods, and food additives cannot generally be identified since it changes with the product, it is preferably chosen from 0.01-7% of range and suitably still more preferably 0.0001 to 10%.

### EXAMPLES

Hereinafter, the present invention is described in more detail by reference to the Examples, which, however, are not intended to limit the present invention.

### Example 1

A commercial labdanum absolute (Givaudan Co., Ltd.) was subjected to molecular distillation. The labdanum absolute (10 g) was subjected to molecular distillation under reduced pressure (13.3 Pa) to collect a fraction (4.3 g) at 180 to 220° C. This fraction contains a mixture of labd-8-en-15-oic acid, labd-7-en-15-oic acid and labd-8(17)-en-15-oic acid (this mixture is referred to hereinafter as axt-1).

### Example 2

### Synthesis of Labdenoic Derivatives from Manool

(A) Production of Primary Allylic Alcohol Represented by Formula (II)

Under a nitrogen atmosphere, 235.4 g of manool, 95.2 g of boric acid, 264.3 g of 1-butanol, 75 g of toluene and 7.5 g of ammonium metavanadate were charged into a reaction flask equipped with a thermometer and a Dean-Stark tube. Under stirring, this solution was added to a 15 g water solution of 1.5 g of sodium carbonate. Heating was started and reaction temperature was increased to 140° C. with azeotropic dehydration, then stirring for 16 hours. After cooling, 311 g of 20% aqueous NaOH was added thereto, the mixture was stirred for 1.5 hours at 60° C., then separated. Then, 1-butanol and toluene were evaporated by heating in vacuo, 1,2,4-trimethylbenzene was added to the residue, and the organic layer was washed 4 times each with 250 mL of water. Thus, 675 g of a 1,2,4-trimethylbenzene solution of primary allylic alcohol represented by formula (2) was obtained in a yield of 70.0% as determined by HPLC analysis. Said primary allyl alcohol represented by formula (2) was used in the next reaction without purification.

(B) Production of Aldehyde Compound Represented by Formula (III)

Under a nitrogen atmosphere, 675 g of the 1,2,4-trimethylbenzene solution of allylic alcohol obtained in (A), 1.71 g of [RuCl₂(p-cymene)] ₂ and 23.5 g of tris(4-methoxyphenyl)phosphine were charged into a reaction flask equipped with a thermometer and a Dean-Stark tube. Under stirring, heating of this solution was started, reaction temperature was increased to 170 to 180° C. for 2 hours, followed by cooling to 46° C., considered as the end of reaction. Thus, 690 g of a 1,2,4-trimethylbenzene solution of an aldehyde compound represented by formula (3) was obtained in a yield of 62.0% as determined by HPLC analysis. Said aldehyde compound represented by formula (3) was used in the next reaction without purification.

(C) Production of Labdenoic Derivatives Represented by Formula (I)

690 g of the 1,2,4-trimethylbenzene solution of aldehyde compound obtained in (B), 300 g of 1,2,4-trimethylbenzene, 0.2 g of acetic acid, 54.57 g of amidosulfuric acid and 27.28 g of water were charged into a reaction flask equipped with a thermometer, then the mixture was cooled to −5° C. by a dry ice/acetone bath under stirring. Then, to this solution was added dropwise 63.54 g of 80% NaClO₂ in 190.6 g of water at −8 to −4° C. within 100 minutes. After stirring for 2 hours at this temperature, to the reaction mixture was added dropwise 425 g of 20% aqueous solution of Na₂SO₃ at −5 to −3° C. within 30 minutes. Then, it was stirred at 40 to 50° C. for 30 minutes, and the peroxide was completely decomposed. After separation, the organic layer was washed 2 times each with 250 g of 5% brine to obtain 980 g of a 1,2,4-trimethylbenzene solution of crude labdenoic derivatives. Said crude solution was added to 73.1 g of 28% MeONa methanol solution for salination of sodium labdenate. Then 150 g of water was added to this mixture, and it separated into an upper neutral layer and a lower layer of the sodium labdenate. After the upper layer was separated, the lower layer was washed 2 times each with 200 mL of heptane. Said washed lower layer was added to 200 mL of heptane and 94.3 g of 20% sulfuric acid for conversion of the labdenoic derivatives from sodium salts, followed by extraction as a heptane layer. The obtained heptane solution was evaporated, and the residue was distilled under reduced pressure to obtain 95.0 g of labdenoic derivatives represented by formula (I) with 92% chemical purity (this mixture of labdenoic derivatives is referred to hereinafter as Syn-1).

Test Example

Test of Acceleration of Collagen Producing Activity

NB1RGB cell lines derived from normal human skin fibroblast (hereinafter referred to as “cells”) were suspended in DMEM containing 10% fetal bovine serum (hereinafter referred to as “FBS”) and seeded into a 96-well plate at a concentration of 20,000 cells/well, then incubated in a CO₂ incubator (37° C., 5% CO₂) for 24 hours. Next, the DMEM was replaced with DMEM containing 0.5% FBS and the accelrator of collagen production obtained in the above, and incubation was further carried out for 5 days. After completion of the incubation, the supernatant was recovered and the cells left in the plate were washed with PBS(−) then the number of cells was determined by neutral red uptake assay.

Collagen production ability of a cell was carried out by measuring the amount of I type procollagen C end peptide (Procollagen type I C-peptide, hereinafter referred to as PIP) secreted in a culture-medium supernatant fluid by the ELISA method. The amount of PIP(s) per the number of cells was calculated, and the relative quantity which makes the control 100% was estimated. The result is shown in the following Table 1 and 2.

TABLE 1
Acceleration of collagen production activity of Ext-1

| | collagen production |
|---|---|
| Concentration | activity (%) |
| Control | 100 |
| 1.56 ppm | 194 |
| 3.13 ppm | 240 |

TABLE 2
Acceleration of collagen production activity of Syn-1

| | collagen production |
|---|---|
| Concentration | activity (%) |
| Control | 100 |
| 1.56 ppm | 244 |
| 3.13 ppm | 372 |

As shown in Table 1 and 2, Ext-1 obtained in Example 1 and Syn-1 obtained in Example 2 showed an acceleration of collagen production of NB1RGB fibroblasts.

### Example 3

According to a conventional method, the accelerator of collagen production of the present invention was used to prepare a cream, emulsion, ointment, tooth paste and mouthwash, respectively.

(1) Cream

TABLE 3

| | Incorporation amount |
|---|---|
| Ingredients | (% by weight) |
| Stearic acid | 6.0 |
| Sorbitan monostearate | 2.0 |
| Polyoxyethylene sorbitan monostearate | 1.5 |
| Propyleneglycol | 10.0 |
| Ext-1 obtained in Example 1 | 1.0 |
| Glycerine trioctanoate | 10.0 |
| Squarene | 5.0 |
| Sodium bisulfite | 0.01 |
| Ethyl p-hydroxybenzoate | 0.3 |
| Perfume | suitable amount |
| Purified water | Adjusted to 100% |

(2) Emulsion

TABLE 4

| | Incorporation amount |
|---|---|
| Ingredients | (% by weight) |
| Stearic acid | 2.5 |
| Cetyl alcohol | 1.5 |
| Vaseline | 5.0 |
| Liquid paraffin | 10.0 |
| Polyoxyethylene monooleate | 2.0 |
| Polyethylene glycol 1500 | 3.0 |
| Triethanolamine | 1.0 |
| Syn-1 obtained in Example 2 | 0.1 |
| Sodium bisulfite | 0.01 |
| Ethyl p-hydroxybenzoate | 0.3 |
| Perfume | suitable amount |
| Purified water | Adjusted to 100% |

(3) Ointment

TABLE 5

| | Incorporation amount |
|---|---|
| Ingredients | (% by weight) |
| Polyoxyethylene cetylether | 5.0 |
| Glycerine monostearate | 10.0 |
| Liquid paraffin | 10.0 |
| Vaseline | 40.0 |
| Cetyl alcohol | 6.0 |
| Methyl p-hydroxybenzoate | 0.1 |
| Butyl p-hydroxybenzoate | 0.1 |
| Glycerine monostearate | 2.0 |
| Ext-1 obtained in Example 1 | 2.0 |
| Propylene glycol | 10.0 |
| Perfume | suitable amount |
| Purified Water | Adjusted to 100% |

(4) Tooth paste

TABLE 6

| | Incorporation amount |
|---|---|
| Ingredients | (% by weight) |
| Calcium carbonate | 3.0 |
| Propylene glycol | 3.0 |
| Sorbitol | 35.0 |
| Sodium lauryl sulfate | 1.5 |
| Carboxy-methyl cellulose | 1.5 |
| Saccharin sodium | 0.1 |
| Methyl p-hydroxybenzoate | 0.1 |
| Syn-1 obtained in Example 2 | 0.5 |
| Perfume | suitable amount |
| Purified water | Adjusted to 100% |

(5) Mouthwash

TABLE 7

| | Incorporation amount |
|---|---|
| Ingredients | (% by weight) |
| Ethanol | 45.0 |
| Glycerine | 51.5 |
| L-Ascorbic acid | 2.0 |
| Sodium lauryl sulfate | 1.0 |
| Sodium citrate | 0.2 |
| Saccharin sodium | 0.05 |
| Sodium benzoate | 0.2 |
| Ext-1 obtained in Example 1 | 0.4 |
| L-Menthol | 0.05 |
| Purified Water | Adjusted to 100% |

According to the present invention, it was revealed that labdenoic acids represented by general formula (I) or salts thereof have an excellent accelerating activity on the production of collagen. These labdenoic acids can be used not only as an agent for external application onto the skin, which is effective for prevention and treatment of wrinkles and flabby skin, but also as an oral cavity composition, which is effective for recovery of collagen of gingival. These labdenoic acids can be incorporated into various items such as conditioners, cream, lotions, skin milk, emulsions, face packs, ointments, tooth paste and mouthwash, etc.

1. A method for accelerating collagen production in a living organism comprising contacting the organism with a compound represented by formula (I) or a salt thereof 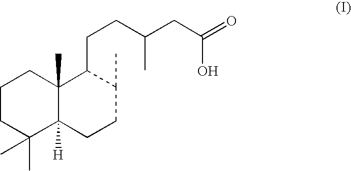
wherein the three dotted lines represent two single bonds and one double bond.

2. The method of claim 1, wherein the compound represented by formula (I) is obtainable from an extract of a cistaceous plant.

3. The method of claim 2, wherein the cistaceous plant is selected from the group consisting of Cistus ladaniferus L., Cistus creticus L., Cistus monoperiensis L. and Cistus salvifoliud.

4. The method of claim 1, wherein the compound represented by formula (I) is synthesized by a chemical process.

5. The method of claim 4, wherein the compound represented by formula (I) is synthesized from sclareol or manool.

6. An agent for external application onto the skin containing a compound represented by formula (I) or a salt thereof and an excipient suitable for topical application: 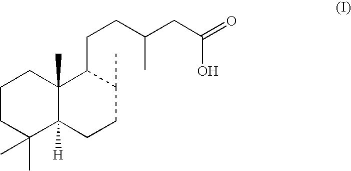
wherein the three dotted lines represent two single bonds and one double bond.

7. An oral cavity composition containing a compound represented by formula (I) or a salt thereof and a pharmaceutically acceptable oral carrier: 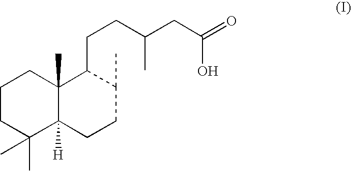
wherein the three dotted lines represent two single bonds and one double bond.
